(19) United States
(12) Patent Application Publication
(10) Pub. No.: US 2017/0342407 A1
(43) Pub. Date: Nov. 30, 2017

(54) IDENTIFYING GENES INVOLVED IN ANTIBIOTIC RESISTANCE AND SENSITIVITY IN BACTERIA USING MICROCULTURES

(71) Applicant: DISCUVA LIMITED, Cambridge (GB)

(72) Inventors: David Hugh Williams, Melbourn (GB); John Richard Wain, Melbourn (GB); Stuart Robert Wood, Longstanton (GB)

(73) Assignee: DISCUVA LIMITED, Cambridge (GB)

(21) Appl. No.: 15/617,797

(22) Filed: Jun. 8, 2017

Related U.S. Application Data
(63) Continuation of application No. PCT/GB2015/053770, filed on Sep. 12, 2015.

(30) Foreign Application Priority Data
Sep. 12, 2014 (GB) 1421854.9

(51) Int. Cl. C12N 15/10; C12Q 1/18; C12Q 1/68

(52) U.S. Cl. CPC C12N 15/1082; C12Q 1/6876; C12Q 1/18; C12Q 2600/156

(57) ABSTRACT

Described is a method for identifying a gene which mediates antibiotic sensitivity or resistance in a target bacterium, the method comprising the steps of: (a) generating a pool of mutant target bacteria by transposon mutagenesis with an activating transposon (TnA), wherein the TnA comprises an outward-facing promoter (TnAP) capable of increasing transcription of a gene at or near its insertion site in the DNA of said target cells; (b) generating a control microdroplet library by encapsulating individual members of the pool of step (a) in microdroplets, the microdroplets comprising a volume of aqueous growth media suspended in an immiscible carrier liquid, each microdroplet comprising a single mutant target cell; (c)generating a test microdroplet library by encapsulating individual members of the pool of step (a) in microdroplets, the microdroplets comprising a volume of aqueous growth media containing the antibiotic and suspended in an immiscible carrier liquid, each microdroplet comprising a single mutant target cell; (d) incubating the control and test microdroplet libraries to produce control and test microcultures; and (e) comparing the distribution of TnA insertions between control and test microcultures to identify a gene which mediates antibiotic sensitivity or resistance in said target bacterium.

### RELATED APPLICATIONS

This application is a continuation of and claims the benefit of International Application No. PCT/GB2015/053770, with an international filing date of Dec. 9, 2015, and entitled “IDENTIFYING GENES INVOLVED IN ANTIBIOTIC RESISTANCE AND SENSITIVITY IN BACTERIA USING MICROCULTURES”, which was published under PCT Article 21(2) in English, and which claims priority to United Kingdom application 1421854.9, filed on Dec. 9, 2014, the entire contents of each of which are incorporated by reference herein.

### FIELD OF THE INVENTION

The present invention relates to methods for identifying genes mediating antibiotic sensitivity or resistance in bacteria, to methods for identifying antibiotics and to processes for producing antibiotics and pharmaceutical compositions comprising said antibiotics.

### BACKGROUND TO THE INVENTION

There is an urgent need for new antibiotics to counter the emergence of new pathogens and resistance to existing antimicrobial drugs. The identification of the targets of candidate antibiotics is critical, since such information can provide access to a large number of functionally related novel drug families. For example, the discovery of the penicillin-binding proteins as targets of penicillin led to the development of a large family of antibiotics, including multiple generations of cephalosporins, penicillins and carbapenems (see Schmid (2006) Nature Biotechnology 24(4): 419-420).

Transposon directed insertion-site sequencing (TraDIS—see Langridge et al. (2009) Genome Research 19: 2308-2316) has recently been described and used to identify: (a) essential genes; (b) genes advantageous (but not essential) for growth; (c) genes disadvantageous for growth under particular conditions; and (d) genes involved in conferring tolerance to certain conditions (“niche-specific” essential genes). Similar techniques have been described in e.g. Gawronski et al. (2009) PNAS 106: 16422-16427; Goodman et al. (2009) Cell Host Microbe 6: 279-289; van Opijnen et al. (2009) Nat. Methods 6: 767-772 and Gallagher et al. (2011) mBio 2(1):e00315-10, and such techniques are now collectively dubbed “Tn-seq” methods.

However, an important class of antibiotic targets are gene products involved in cellular processes essential for viability in the growth conditions used. Such targets cannot be identified by Tn-seq (including TraDIS), since transposon insertions into essential genes (including those serving as antibiotic targets) are not significantly represented in the initial

mutant pool. Thus, differences in transposon distribution after growth of the mutant pool with or without (or with varying amounts of) antibiotic would not arise, with the result that Tn-seq cannot distinguish between an essential gene and an essential gene serving as an antibiotic target.

There is therefore a need for high-throughput functional screens for antibiotic targets which are capable of identifying essential genes serving as antibiotic targets.

WO2012/150432 describes a method for identifying an essential gene which serves as an antibiotic target in a bacterium comprising the steps of:
(a) generating a pool of mutant bacteria by transposon mutagenesis with an activating transposon (Tn_{A}), wherein the Tn_{A} comprises a promoter such that transposon insertion into bacterial DNA increases the transcription of a gene at or near the insertion site;
(b) growing bacteria from the mutant pool in the presence of different amounts of said antibiotic to produce two or more test cultures; and
(c) comparing the distribution of Tn_{A} insertions between test cultures to identify a putative essential gene serving as a target of said antibiotic in said bacterium.

It has now been discovered that the quality of the quantitative sequencing data obtained via such methods is greatly improved and enriched by partitioning individual mutant bacteria from the mutant pool into separate microcultures prior to the growing step.

Without wishing to be bound by any theory, it is thought that mutant bacteria containing Tn_{A} insertions in genes which mediate antibiotic sensitivity or resistance display a wide range of fitness, to the extent that conventional co-culture of the mutants in bulk liquid culture results in surprisingly high losses of antibiotic resistant mutants of interest which are “swamped” by faster growing mutants. It is believed that the culturing each mutant in its own discrete micro-environment effectively eliminates signal loss arising from out-competition by faster growing mutants, greatly increasing the resolution of the technology.

Moreover, the microdroplet cultures can be left to grow for periods sufficiently long so as to permit even the slowest growing mutants to complete many doublings (typically up to 7 days), without any loss of library diversity arising from overgrowth by relatively fast growing mutants (which would inevitably occur in bulk liquid cultures over such periods).

### SUMMARY OF THE INVENTION

According to an aspect of the present invention, there is provided a method for identifying a gene which mediates antibiotic sensitivity or resistance in a target bacterium, the method comprising the steps of:
(a) generating a pool of mutant target bacteria by transposon mutagenesis with an activating transposon (Tn_{A}), wherein the Tn_{A} comprises an outward-facing promoter (Tn_{A}P) capable of increasing transcription of a gene at or near its insertion site in the DNA of said target cells;
(b) generating a control microdroplet library by encapsulating individual members of the pool of step (a) in microdroplets, the microdroplets comprising a volume of aqueous growth media suspended in an immiscible carrier liquid, each microdroplet comprising a single mutant target cell;
(c) generating a test microdroplet library by encapsulating individual members of the pool of step (a) in microdroplets, the microdroplets comprising a volume of aqueous growth media containing the antibiotic and suspended in an immiscible carrier liquid, each microdroplet comprising a single mutant target cell;
(d) incubating the control and test microdroplet libraries to produce control and test microcultures; and
(e) comparing the distribution of Tn_{A} insertions between control and test microcultures to identify a gene which mediates antibiotic sensitivity or resistance in said target bacterium.

The method of the invention avoids the “swamping” effect of mutant target bacteria which are resistant to the antibiotic and which have high growth rates relative to other mutants of interest in which the Tn_{A} insertion has led to a significant loss of fitness. Such “weakened” mutants would be overgrown (and their contribution to the TnA insertion distribution dataset depleted or extinguished) if not effectively partitioned as individual mutants by the encapsulation step of the invention.

The encapsulation step is preferably conducted such that each microdroplet contains a single member of the mutant pool. Depending on the encapsulation process employed, the microdroplet library may be heterogeneous with respect to cellular content, and some microdroplets may be empty. However, all that is required is that encapsulation result in the recovery of at least some microdroplets containing a single mutant target cell from the mutant pool.

The size of the microdroplets will be selected by reference to the nature of the target cells to be encapsulated, and the number of doublings to be achieved during the incubation step. Typically, the microdroplets are sized to provide a volume of growth medium sufficient to support 1000 cells. Thus, the microdroplets may be substantially spherical with a diameter of: (a) 10 μm to 500 μm; (b) 10 μm to 200 μm; (c) 10 μm to 150 μm; (d) 10 μm to 100 μm; (e) 10 μm to 50 μm; or (f) about 100 μm.

While the microdroplets may comprise a volume of aqueous growth media in the gel state, in preferred embodiments they comprise a volume of aqueous growth media in the liquid state. In such embodiments, the microdroplets may comprise an inner core of aqueous growth media enveloped in an outer oil shell, the carrier liquid being a continuous aqueous phase. Here, the inner aqueous core has a diameter of: (a) 10 μm to 500 μm; (b) 10 μm to 200 μm; (c) 10 μm to 150 μm; (d) 10 μm to 100 μm; (e) 10 μm to 50 μm; or (f) about 100 μm, while the outer oil shell may have a thickness of: (a) 10 μm to 200 μm; (b) 10 μm to 200 μm; (c) 10 μm to 150 μm; (d) 10 μm to 100 μm; (e) 10 μm to 50 μm; or (f) about 100 μm.

In single W/O type emulsions, the carrier liquid may be any water-immiscible liquid, for example an oil, optionally selected from: (a) a hydrocarbon oil; (b) a fluorocarbon oil; (c) an ester oil; (d) an oil having low solubility for biological components of the aqueous phase; (e) an oil which inhibits molecular diffusion between microdroplets; (f) an oil which is hydrophobic and lipophobic; (g) an oil having good solubility for gases; and/or (h) combinations of any two or more of the foregoing.

Thus, the microdroplets may be comprised in a W/O emulsion wherein the microdroplets constitute an aqueous, dispersed, phase and the carrier liquid constitutes a continuous oil phase.

In other embodiments, the microdroplets are comprised in a W/O/W double emulsion and the carrier liquid may an aqueous liquid. In such embodiments, the aqueous liquid may be phosphate buffered saline (PBS).

The microdroplets may therefore be comprised in a W/O/W double emulsion wherein the microdroplets comprise: (a) an inner core of aqueous growth media enveloped in an outer oil shell as the dispersed phase, and (b) the carrier liquid as the continuous aqueous phase.

In embodiments where the microdroplets are comprised in an emulsion, the carrier liquid may constitute the continuous phase and the microdroplets the dispersed phase, and in such embodiments the emulsion may further comprise a surfactant and optionally a co-surfactant.

The surfactant and/or co-surfactant may be located at the interface of the dispersed and continuous phases, and when the microdroplets are comprised in a W/O/W double emulsion the surfactant and/or co-surfactant may be located at the interface of aqueous core and oil shell and at the interface of the oil shell and outer continuous phase

The microdroplets may be monodispersed (as defined herein).

The encapsulation steps may comprise mixing: (i) the pool of mutant target cells; (ii) an aqueous growth medium; (iii) a water-immiscible liquid, for example an oil as defined herein; and (iv) a surfactant, for example as defined herein, under conditions whereby a W/O type single emulsion comprising microdroplets of the aqueous growth medium dispersed in the water-immiscible liquid is formed.

In some embodiments, the W/O type single emulsion as described above is used for the incubation step. This may be preferred in circumstances where the continuous oil phase provides improved compartmentalization of the microcultures (for example, by preventing or limiting inter-culture interactions mediated by water soluble bioactive produces released during culture). In such embodiments, subsequent manipulation, screening (and in particular sorting) may be facilitated by a further, post-incubation, emulsification step wherein an aqueous carrier liquid, for example as defined herein, is mixed with the single emulsion used for the incubation step under conditions whereby a W/O/W double emulsion comprising microdroplets of the aqueous growth medium enveloped in the water-immiscible liquid and dispersed in the aqueous carrier liquid is formed.

The encapsulation steps may comprises mixing: (i) the pool of mutant target cells; (ii) an aqueous growth medium; (iii) a water-immiscible liquid, for example an oil as defined herein; (iv) a surfactant, for example as defined herein, and (v) an aqueous carrier liquid, for example as defined herein, under conditions whereby a W/O/W double emulsion comprising microdroplets of the aqueous growth medium enveloped in the water-immiscible liquid and dispersed in the aqueous carrier liquid is formed.

Any means may be employed for the mixing step: for example, this step may comprise: (a) vortexing and/or (b) sonication; (c) homogenization; (d) pico-injection and/or (e) flow focusing.

As explained above, depending on the encapsulation process employed, the microdroplet library may be heterogeneous with respect to cellular content, and some microdroplets may be empty. In such circumstances the encapsulation steps may further comprise eliminating empty microdroplets which do not contain mutant target cells. This step is conveniently achieved by Fluorescence-Activated Droplet Sorting (FADS), and in such embodiments the target cells are fluorescently labelled.

The incubation step (d) is carried out for a period and under conditions selected by reference to the nature of the target cells. Thus, this step may comprise maintaining the microdroplet library at a suitable temperature for at least 1 hour.

The incubation step (d) may comprise maintaining the microdroplet library at said temperature for about 2, 4, 6, 12, 24 or 48 hours, or for up to 7 days, for example for 1, 2, 3, 4, 5, 6 or 7 days. In other embodiments, the incubation step (d) comprises maintaining the microdroplet library at said temperature for up to 2 weeks, for example for 1 week or 2 weeks.

Other aspects and preferred embodiments of the invention are defined and described in the other claims set out below.

### BRIEF DESCRIPTION OF THE DRAWINGS

FIG. 1 shows the results of exposure to ciprofloxacin (EC₉₅) in E. coli cells mutagenized with an activating transposon (TnA) as described in WO2012/150432. Part A shows the proportions of the four different mutant classes when grown in standard open broth culture. Part B shows the proportions of the four different mutant classes when grown in microdroplets.

### DETAILED DESCRIPTION OF THE INVENTION

All publications, patents, patent applications and other references mentioned herein are hereby incorporated by reference in their entireties for all purposes as if each individual publication, patent or patent application were specifically and individually indicated to be incorporated by reference and the content thereof recited in full.

### Definitions and General Preferences

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

The term gene is a term describing a hereditary unit consisting of a sequence of DNA that occupies a specific location on a chromosome or plasmid and determines a particular characteristic in an organism. A gene may determine a characteristic of an organism by specifying a polypeptide chain that forms a protein or part of a protein (structural gene); or encode an RNA molecule; or regulate the operation of other genes or repress such operation; or affect phenotype by some other as yet undefined mechanism.

The terms genomic DNA is a term of art used herein to define chromosomal DNA as distinct from extrachromosomally-maintained plasmid DNA.

The term genome is a term of art used herein to define the entire genetic complement of an organism, and so includes chromosomal, plasmid, prophage and any other DNA.

The term Gram-positive bacterium is a term of art defining a particular class of bacteria that are grouped together on the basis of certain cell wall staining characteristics.

The term low G+C Gram-positive bacterium is a term of art defining a particular subclass class of evolutionarily related bacteria within the Gram-positives on the basis of the composition of the bases in the DNA. The subclass includes Streptococcus spp., Staphylococcus spp., Listeria spp., Bacillus spp., Clostridium spp., Enterococcus spp. and Lactobacillus spp.).

The term high G+C Gram-positive bacterium is a term of art defining a particular subclass class of evolutionarily related bacteria within the Gram-positives on the basis of the composition of the bases in the DNA. The subclass includes actinomycetes (actinobacteria) including Actinomyces spp., Arthrobacter spp., Corynebacterium spp., Frankia spp., Micrococcus spp., Micromonospora spp., Mycobacterium spp., Nocardia spp., Propionibacterium spp. and Streptomyces spp.

The term Gram-negative bacterium is a term of art defining a particular class of bacteria that are grouped together on the basis of certain cell wall, staining characteristics. Examples of Gram-negative bacterial genera include Klebsiella, Acinetobacter, Escherichia, Pseudomonas, Enterobacter and Neisseria.

The term “monodisperse” as applied to the microdroplets means any emulsion showing a coefficient of particle size dispersion, ε, of not more than 1.0, not more than 0.5, and preferably not more than 0.3. Said coefficient ε is defined by the following equation:

ε=(⁹⁰Dₚ−¹⁰Dₚ)/⁵⁰Dₚ(1)

where ¹⁰Dₚ, ⁵⁰Dₚ and ⁹⁰Dₚ are the particle sizes when the cumulative frequencies estimated from a relative cumulative particle size distribution curve for the emulsion are 10%, 50% and 90%, respectively. The case where ε=0 means an ideal state in which emulsion particles show no particle size scattering at all.

As used herein, the term “insertion rate” as applied to transposon insertion, is used to indicate the density of Tn_{A} insertion at the level of the mutant pool as a whole, with one Tn_{A} insertion in each bacterium. It will also be understood that lethal Tn_{A} insertion events, such as those arising from insertional inactivation of an essential gene, will not be represented by viable members of the mutant pool. Thus, the insertion rates specified herein apply to non-essential regions of the DNA.

Antibiotics and Antibiotic Targets

The antibiotic used to produce the test cultures of the invention is typically a novel investigational antibiotic (anti-bacterial chemotherapeutic agent), the mechanism of action (and hence biological target(s)) of which are unknown. In many applications, the antibiotic is selected from combinatorial libraries, natural product libraries, defined chemical entities, peptides, peptide mimetics and oligonucleotides.

The antibiotic target identified according to the invention is an essential gene/gene product, and may therefore be involved in one or more of the following biological processes in the bacterial host:
(a) cell division;
(b) DNA replication (including polymerization and supercoiling);
(c) transcription (including priming, elongation and termination);
(d) translation (including ribosome components, initiation, elongation and release);
(e) biosynthetic pathways (including peptidoglycan and fatty acids);
(f) plasmid addiction;
(g) cell wall assembly; and/or
(h) bacterial cell integrity.

Target Bacteria for Use in the Methods of the Invention

The methods of the invention may be applied to identify an antibiotic target in any bacterium. Thus, the methods of the invention find application in the identification of antibiotic targets in: (a) Gram-positive, Gram-negative and/or Gram-variable bacteria; (b) spore-forming bacteria; (c) non-spore forming bacteria; (d) filamentous bacteria; (e) intracellular bacteria; (f) obligate aerobes; (g) obligate anaerobes; (h) facultative anaerobes; (i) microaerophilic bacteria and/or (f) opportunistic bacterial pathogens.

In certain embodiments, the methods of the invention are applied to identify an antibiotic target in bacteria of the following genera: Acinetobacter (e.g. A. baumannii); Aeromonas (e.g. A. hydrophila); Bacillus (e.g. B. anthracis); Bacteroides (e.g. B. fragilis); Bordetella (e.g. B. pertussis); Borrelia (e.g. B. burgdorferi); Brucella (e.g. B. abortus, B. canis, B. melitensis and B. suis); Burkholderia (e.g. B. cepacia complex); Campylobacter (e.g. C. jejuni); Chlamydia (e.g. C. trachomatis, C. suis and C. muridarum); Chlamydophila (e.g. (e.g. C. pneumoniae, C. pecorum, C. psittaci, C. abortus, C. felis and C. caviae); Citrobacter (e.g. C. freundii); Clostridium (e.g. C. botulinum, C. difficile, C. perfringens and C. tetani); Corynebacterium (e.g. C. diphteriae and C. glutamicum); Enterobacter (e.g. E. cloacae and E. aerogenes); Enterococcus (e.g. E. faecalis and E. faecium); Escherichia (e.g. E. coli); Flavobacterium; Francisella (e.g. F. tularensis); Fusobacterium (e.g. F. necrophorum); Haemophilus (e.g. H. somnus, H. influenzae and H. parainfluenzae); Helicobacter (e.g. H. pylori); Klebsiella (e.g. K. oxytoca and K. pneumoniae), Legionella (e.g. L. pneumophila); Leptospira (e.g. L. interrogans); Listeria (e.g. L. monocytogenes); Moraxella (e.g. M. catarrhalis); Morganella (e.g. M. morganii); Mycobacterium (e.g. M. leprae and M. tuberculosis); Mycoplasma (e.g. M. pneumoniae); Neisseria (e.g. N. gonorrhoeae and N. meningitidis); Pasteurella (e.g. P. multocida); Peptostreptococcus; Prevotella; Proteus (e.g. P. mirabilis and P. vulgaris), Pseudomonas (e.g. P. aeruginosa); Rickettsia (e.g. R. rickettsii); Salmonella (e.g. serotypes. Typhi and Typhimurium); Serratia (e.g. S. marcesens); Shigella (e.g. S. flexnaria, S. dysenteriae and S. sonnei); Staphylococcus (e.g. S. aureus, S. haemolyticus, S. intermedius, S. epidermidis and S. saprophyticus); Stenotrophomonas (e.g. S. maltophila); Streptococcus (e.g. S. agalactiae, S. mutans, S. pneumoniae and S. pyogenes); Treponema (e.g. T. pallidum); Vibrio (e.g. V. cholerae) and Yersinia (e.g. Y. pestis).

The methods of the invention may be used to identify an antibiotic target in multi-drug resistant bacteria, including, but not limited to penicillin-resistant, methicillin-resistant, quinolone-resistant, macrolide-resistant, and/or vancomycin-resistant bacterial strains, including for example penicillin-, methicillin-, macrolide-, vancomycin-, and/or quinolone-resistant Streptococcus pneumoniae; penicillin-, methicillin-, macrolide-, vancomycin-, and/or quinolone-resistant Staphylococcus aureus; penicillin-, methicillin-, macrolide-, vancomycin-, and/or quinolone-resistant Streptococcus pyogenes; and penicillin-, methicillin-, macrolide-, vancomycin-, and/or quinolone-resistant enterococci.

Thus, methods of the invention may be used to identify an antibiotic target in methicillin-resistant Staphylococcus aureus (MRSA), for example selected from any of C-MSRA1, C-MRSA2; C-MRSA3, C-MSRA4, Belgian MRSA, Swiss MRSA and any of the EMRSA strains.

The compounds of the invention may be used to identify an antibiotic target in both high G+C Gram-positive bacteria and in low G+C Gram-positive bacteria.

The methods of the invention find particular application in the identification of an antibiotic target in a bacterium selected from Klebsiella pneumoniae, Acinetobacter baumanii, Escherichia coli (including ST131), Enterococcus faecalis, Enterococcus faecium, Pseudomonas aeruginosa, Enterobacter cloacae, Enterobacter aerogenes and Neisseria gonorrhoeae.

Particularly preferred are methods of identifying an antibiotic target in Klebsiella pneumoniae, Acinetobacter baumanii or Escherichia coli.

Mutant Pools

The methods of the invention involve generating a pool of mutant bacteria by transposon mutagenesis. The size of the mutant pool affects the resolution of the method: as the pool size increases, more and more different genes with Tn_{A} insertions will be represented (and so effectively assayed). As the pool size decreases, the resolution of the method reduces, genes will be less effectively assayed, and more and more genes will not be assayed at all.

Ideally, the mutant pool generated in the methods of the invention is comprehensive, in the sense that insertions into every gene are represented. The number of Tn_{A} insertion mutants (i.e. the mutant pool size) required to achieve this depends on various factors, including: (a) the size of the bacterial genome; (b) the average size of the genes; and (c) any Tn_{A} insertion site bias.

With regard to the latter, some areas of bacterial genomes attract a low frequency of insertion (especially GC-rich regions). Thus, insertion frequencies and pool sizes large enough to ensure that insertions into insertion-refractory regions are preferred.

In general, a minimum insertion rate of one transposon per 25 bp is required to achieve a comprehensive pool/library, which typically entails a minimum pool size for bacteria having a genome size of 4 to 7 Mb of 0.5×10⁵ to 1×10⁵, for example 5×10⁵, preferably at least about 1×10⁶ mutants. In many cases, 1×10⁶ mutants will allow identification of ˜300,000 different insertion sites and correspond to 1 transposon insertion every 13 to 23 bp (or about 40-70 different insertion sites per gene).

However, the methods of the invention do not necessarily require a comprehensive mutant pool (in the sense defined above) in order to return useful information as to the identity of antibiotic drug targets. Rather, pool sizes less than the ideal comprehensive pool may be used, provided that a reduction in resolution (and attendant failure to assay certain genes) can be tolerated. This may be the case, for example, where the method is designed to be run iteratively until the target is identified: in such embodiments the effective pool size grows with each iteration of the method.

Transposon Mutagenesis

Transposons, sometimes called transposable elements, are polynucleotides capable of inserting copies of themselves into other polynucleotides. The term transposon is well known to those skilled in the art and includes classes of transposons that can be distinguished on the basis of sequence organisation, for example short inverted repeats at each end; directly repeated long terminal repeats (LTRs) at the ends; and polyA at 3′ends of RNA transcripts with 5′ ends often truncated.

Transposomes are transposase-transposon complexes wherein the transposon does not encode transposase. Thus, once inserted the transposon is stable. Preferably, in order to ensure mutant pool stability, the transposon does not encode transposase and is provided in the form of a transposome (i.e. as a complex with transposase enzyme), as described below.

As used herein, the term “activating transposon” (hereinafter abbreviated “Tn_{A}”) defines a transposon which comprises a promoter such that transposon insertion increases the transcription of a gene at or near the insertion site (i.e. an outward-facing promoter functional in the host bacterium). Examples of such transposons are described in Troeschel et al. (2010) Methods Mol Biol. 668:117-39 and Kim et al. (2008) Curr Microbiol. 57(4): 391-394.

The activating transposon/transposome can be introduced into the bacterial genome (including chromosomal and/or plasmid DNA) by any of a wide variety of standard procedures which are well-known to those skilled in the art. For example, Tn_{A} transposomes can be introduced by electroporation (or any other suitable transformation method).

Preferably, the transformation method generates 1×10³ to 5×10³ transformants/ng DNA, and such transformation efficiencies are generally achievable using electroporation.

Alternatively, transposon mutagenesis using Tn_{A} may be performed in vitro and recombinant molecules transformed/transfected into bacterial cells. In such embodiments, transposomes can be prepared according to a standard protocol by mixing commercially available transposase enzyme with the transposon DNA fragment. The resulting transposomes are then mixed with plasmid DNA of the plasmid of interest to allow transposition, then the DNA introduced into a host bacterial strain using electrotransformation to generate a pool of plasmid transposon mutants.

In embodiments where mutagenesis is performed in vitro, it is possible to mix transposomes with genomic DNA in vitro and then introduce the mutagenized DNA (optionally, after fragmentation and/or circularization) into the host bacterial strain (e.g. by electroporation) whereupon endogenous recombination machinery incorporates it into the genome. Such an approach may be particularly useful in the case of bacteria which are naturally competent (e.g. Acinetobacter spp.) and/or can incorporate DNA via homologous crossover (e.g. double crossover) recombination events.

Activating Transposons for Use in the Methods of the Invention

Any suitable activating transposon may be used in the methods of the invention. Suitable transposons include those based on Tn3 and the Tn3-like (Class II) transposons including γδ (Tn1000), Tn501, Tn2501, Tn21, Tn917 and their relatives. Also Tn10, Tn5, TnphoA, Tn903, TN5096, Tn5099, Tn4556, UC8592, IS493, bacteriophage Mu and related transposable bacteriophages. A variety of suitable transposons are also available commercially, including for example the EZ-Tn5™<R6Kγori/KAN-2> transposon.

Preferred transposons are those which carry antibiotic resistance genes although any selectable marker can be used including auxotrophic complimentation (which may be useful in identifying mutants which carry a transposon) including Tn5, Tn10 and TnphoA. For example, Tn10 carries a tetracycline resistance gene between its IS elements while Tn5 carries genes encoding polypeptides conferring resistance to kanamycin, streptomycin and bleomycin: Other suitable resistance genes include those including neomycin, apramycin, thiostrepton and chloramphenicol acetyltransferase (conferring resistance to chloramphenicol).

It is of course possible to generate new transposons by inserting different combinations of antibiotic resistance genes between IS elements, or by inserting combinations of antibiotic resistance genes between transposon mosaic ends (preferred), or by altering the polynucleotide sequence of the transposon, for example by making a redundant base substitution or any other type of base substitution that does not affect the transposition or the antibiotic resistance characteristics of the transposon, in the coding region of an antibiotic resistance gene or elsewhere in the transposon. Such transposons are included within the scope of the invention.

In many embodiments, a single transposon is used to generate the mutant pool. However, as explained above, the number of Tn insertion mutants (i.e. the mutant pool size) required to achieve a comprehensive pool or library depends inter alia on any Tn insertion site bias. Thus, in cases where the transposon insertion site bias occurs, two or more different transposons may be used in order to reduce or eliminate insertion site bias. For example, a combination of two different transposons based on Tn5 and Tn10 may be employed.

Promoters for Use in Activating Transposons

The nature of the promoter present in the Tn_{A} is dependent on the nature of the transposon and the ultimate target cell host. Generally, an efficient, outward-oriented promoter which drives constitutive and/or high level transcription of DNA near or adjacent to the insertion site is chosen.

The promoter may include: (a) a Pribnow box (−10 element); (b) a −35 element and/or (c) an UP element.

For example, the lac promoter can be used with the EZ-Tn5™<R6Kγori/KAN-2> transposon, and such constructs are suitable for assay of e.g. Escherichia coli, Enterobacter spp. and other members of the family Enterobacteriaceae such as Klebsiella spp. Other suitable promoters include: rplJ (large ribosomal subunit protein; moderate strength promoter); tac (artificial lac/trp hybrid; strong promoter) and rrnB (ribosomal RNA gene promoter; very strong promoter).

Suitable promoters can also be engineered or selected as described in Rhodius et al. (2011) Nucleic Acids Research: 1-18 and Zhao et al. (2013) ACS Synth. Biol. 2 (11): 662-669. The rapid application of next generation sequencing to RNA-seq is now providing a wealth of high-resolution information of transcript start sites at a genomic level, which greatly simplifies the identification of promoter sequences in any given prokaryote. This permits the construction of descriptive promoter models for entire genomes. RNA-seq also provides quantitative information on transcript abundance and hence promoter strength, which enables the construction of promoter strength models that can then be used for predictive promoter strength rankings (see Rhodius et al. (2011) Nucleic Acids Research: 1-18).

Thus, real-time PCR and RNA-seq permit the rapid identification of strong constitutive promoters from the upstream regions of the housekeeping genes in the selected target cell.

Suitable promoters can also be identified by assay. For example, a series of plasmids can be constructed to test promoter strength empirically. Briefly, the promoter to be tested is placed upstream of an antibiotic resistance gene and then transformed into the relevant bacteria. General cloning assembly and plasmid amplification can be carried out in E. coli (facilitated by the ampicillin resistance gene and the pBR322 ori) and the activity of the promoter in the target bacterium can then be assayed by generating a killing curve with the relevant antibiotic—a very high level promoter gives more antibiotic resistance expression and therefore survival at a higher antibiotic concentration. The plasmid series is designed to be modular so that the origin of replication, resistance gene(s) and promoter can be easily switched.

Suitable promoters for use with Actinobacteria in general (and Streptomyces spp. in particular) include the actinobacterial gapdh and rpsL promoters described in Zhao et al. (2013) ACS Synth. Biol. 2 (11): 662-669.

In circumstances where multiple promoters of different strengths are to be used (see below for more details in this regard), then gapdh from Eggerthella lenta and rpsL from Cellulomonas flavigina may be used as the basis for very strong promoters, gapdh and rpsL from S. griseus may be used as the basis for medium strength promoters while rpoA and rpoB from S. griseus may be used as the basis for low strength promoters (see Shao et al. (2013) ACS Synth. Biol. 2 (11): 662-669).

Other suitable promoters include ermE*, a mutated variant of the promoter of the erythromycin resistance gene from Saccharopolyspora erythraea (see e.g. Wagner et al. (2009) J. Biotechnol.142: 200-204).

Suitable promoters for use with Actinobacteria may be identified and/or engineered as described in Seghezzi et al. (2011) Applied Microbiology and Biotechnology 90(2): 615-623, where the use of randomised −10 and −35 boxes to identify important sequences for expression levels is described. Another approach is described in Wang et al. (2013) Applied and Environmental Microbiology 79(14): 4484-4492.

Suitable promoters for use with Bacillus spp. May be based on the many different promoters described for the model organism Bacillus subtilis, including for example the P43, amyE and aprE promoters from B. subtilis (see e.g. Kim et al. (2008) Biotechnology and Bioprocess Engineering 13(3) 313-318).

Use of Multiple Tn_{A}/Multiple Promoters

In some embodiments of the invention, the target bacterial genome is probed with a mixture of different activating transposons which have outward facing promoters of different strengths. In some circumstances, a broader range of genes involved in antibiotic resistance and/or sensitivity are recovered if a mixture of activating transposons with at least three different promoters of progressively decreasing strength are employed to generate the mutant pool.

In such circumstances, the use of a plurality of activating transposons with promoters of varying strength ensures that transposon insertions occur substantially in all non-essential genes are represented in the initial mutant pool, since transposon insertion can now result in gene activation to yield an appropriate level of transcription (neither too high, nor too low).

In such embodiments, a wide variety of promoters may be used provided that at least three different promoters are used wherein the relative strength of said promoters is: Tn_{A}P1>Tn_{A}P2>Tn_{A}P3; such that transposon insertion into bacterial DNA generates a pool of mutant cells containing members in which one or more genes are transcribed from Tn_{A}P1, one or more genes are transcribed from Tn_{A}P2 and one or more genes are transcribed from Tn_{A}P3.

Preferably, Tn_{A}P1 is a strong promoter, Tn_{A}P2 a medium-strength promoter and Tn_{A}P3 a weak promoter in the mutagenized bacterial host cells under the conditions used for incubation and culture of the mutant pool in the presence of the target cells. In some embodiments, the relative transcription initiation rate of Tn_{A}P1 is at least 3 times, at least 100 times, at least 1000 times or at least 10000 times higher than that of Tn_{A}P3 under these conditions.

Each promoter typically includes: (a) a Pribnow box (−10 element); (b) a −35 element and (c) an UP element. Those skilled in the art are able to readily identify promoters having the required relative strengths by sequence analysis and/or in vitro or in vivo assays using expression constructs.

Suitable promoters include the E. coli rplJ (large ribosomal subunit protein; moderate strength promoter); tac (artificial lac/trp hybrid; strong promoter) and rrnB (ribosomal RNA gene promoter; very strong promoter) promoters.

As used herein, the terms P_{rplJ} and P_{rrnB} specifically refer to the E. coli promoters for the 50S ribosomal subunit protein L10 and 16S ribosomal RNA genes, respectively. Orthologues of these (and other) E. coli promoters from other Gram-negative bacteria can also be used, including in particular the orthologous Pseudomonas aeruginosa or Acinetobacter baumannii promoters.

For example, the orthologous Acinetobacter baumannii gene corresponding to the E. coli rrnB P_{rrnB} has the gene symbol A1S_r12 and encodes the Acinetobacter baumannii 16S ribosomal RNA gene, so that the corresponding orthologous promoter is herein designated P_{(A1S}_{_}ᵣ₁₂₎. Thus, when the method is applied to Acinetobacter baumannii, Tn_{P1} may be P_{(A1S}_{_}ᵣ₁₂₎.

Similarly, when the method of the invention is applied to Pseudomonas aeruginosa, Tn_{P2} may be the 16S ribosomal RNA gene promoter from P. aeruginosa (i.e. Ps.P_{rrnB}) 1 while Tn_{P3} may be selected from the rpsJ (small (30S) ribosomal subunit S10 protein) gene promoter from P. aeruginosa (i.e. Ps.P_{rpsJ}) and the E. coli P_{rrnB}.

Effects of Tn_{A} Insertion

The use of transposon mutagenesis with an activating transposon (Tn_{A}) according to the methods of the invention is capable of producing an extremely diverse range of phenotypes, since the effect of insertion of the Tn_{A} can vary from total loss of function, decreased activity, to varying degrees of increased activity, with change of function (and even gain of function) also being possible.

This follows from the fact that the effect of insertion of the Tn_{A} into the DNA of the bacterial target cell is sequence context dependent. Insertion into the coding sequence of a structural gene may result in insertional inactivation (and so complete loss of function), while insertion upstream of a gene or operon can result in Tn_{A}P driving increased transcription and so lead to expression of that gene or operon (with the extent of overexpression being dependent on subtle positional/polar effects).

A further layer of complexity is introduced by the fact that Tn_{A} insertion may result in transcripts from both sense and anti-sense strands, resulting in the production of antisense transcripts arising from Tn_{A}P driving transcription of the non-coding strand of the DNA of the mutant target cell. Such antisense transcripts may suppress or activate gene expression (for example they may suppress expression of genes by binding to complementary mRNA encoded by the corresponding coding (sense) strand of the DNA of said target cell), or may activate gene transcription by suppressing expression of genes encoding gene repressors.

Determining the Sequence Context of Tn_{A} Insertions

As explained above, the use of transposon mutagenesis with an activating transposon (Tn_{A}) according to the methods of the invention results in the production of a richly diverse mutant pool.

This aspect of the invention is complemented by the ability to establish the sequence context of transposon insertions associated with the phenotype of interest.

The sequence context is preferably determined by sequencing DNA adjacent or near (5′ and/or 3′) the Tn_{A} insertion site (e.g. by sequencing DNA which comprises Tn_{A}-genomic DNA junctions). Typically, bacterial DNA flanking or adjacent to one or both ends of the Tn_{A} is sequenced.

The length of adjacent DNA sequenced need not be extensive, and is preferably relatively short (for example, less than 200 base pairs).

Various methods can be used to determine the Tn_{A} insertion distribution using DNA sequencing: such methods have recently been dubbed Tn-seq procedures (van Opijnen et al. (2009) Nat. Methods 6: 767-772). For example, Tn-seq procedures include affinity purification of amplified Tn junctions (Gawronski et al. (2009) PNAS 106: 16422-16427); ligation of adaptors into genome sequences distal to the end of the transposon using a specialized restriction site (Goodman et al. (2009) Cell Host Microbe 6: 279-289; van Opijnen et al. (2009) Nat. Methods 6: 767-772); selective amplification (Langridge et al. (2009) Genome Research 19: 2308-2316) and the generation of single-stranded DNA circles bearing Tn junctions, which serve as templates for amplification and sequencing after elimination of genomic DNA by exonuclease digestion (Gallagher et al. (2011) mBio 2(1):e00315-10).

Any suitable high-throughput sequencing technique can be used, and there are many commercially available sequencing platforms that are suitable for use in the methods of the invention. Sequencing-by-synthesis (SBS)-based sequencing platforms are particularly suitable for use in the methods of the invention: for example, the Illumina™ system is generates millions of relatively short sequence reads (54, 75 or 100 bp) and is particularly preferred.

Other suitable techniques include methods based on reversible dye-terminators. Here, DNA molecules are first attached to primers on a slide and amplified so that local clonal colonies are formed (bridge amplification). Four types of ddNTPs are added, and non-incorporated nucleotides are washed away. Unlike pyrosequencing, the DNA can only be extended one nucleotide at a time. A camera takes images of the fluorescently labeled nucleotides then the dye along with the terminal 3′ blocker is chemically removed from the DNA, allowing a next cycle.

Other systems capable of short sequence reads include SOLiD™ and Ion Torrent technologies (both sold by Applied Biosystems™) SOLiD™ technology employs sequencing by ligation. Here, a pool of all possible oligonucleotides of a fixed length are labeled according to the sequenced position. Oligonucleotides are annealed and ligated; the preferential ligation by DNA ligase for matching sequences results in a signal informative of the nucleotide at that position. Before sequencing, the DNA is amplified by emulsion PCR. The resulting bead, each containing only copies of the same DNA molecule, are deposited on a glass slide. The result is sequences of quantities and lengths comparable to Illumine sequencing.

Ion Torrent Systems Inc. have developed a system based on using standard sequencing chemistry, but with a novel, semiconductor based detection system. This method of sequencing is based on the detection of hydrogen ions that are released during the polymerisation of DNA, as opposed to the optical methods used in other sequencing systems. A microwell containing a template DNA strand to be sequenced is flooded with a single type of nucleotide. If the introduced nucleotide is complementary to the leading template nucleotide it is incorporated into the growing complementary strand. This causes the release of a hydrogen ion that triggers a hypersensitive ion sensor, which indicates that a reaction has occurred. If homopolymer repeats are present in the template sequence multiple nucleotides will be incorporated in a single cycle. This leads to a corresponding number of released hydrogens and a proportionally higher electronic signal.

Functional Assessment of Putative Essential Genes

The putative essential gene identified by comparing the distribution of Tn_{A} insertions between test cultures may be further characterized by various techniques which directly or indirectly assess its function. In this way, an essential function may be definitively assigned to said putative essential gene.

Suitable techniques include bioinformatics, where the (full or partial) sequence of the putative essential gene is used to interrogate sequence databases containing information from the bacterium assayed and/or other species in order to identify genes (e.g. orthologous genes in other species) for which essential biochemical function(s) have already been assigned and/or which have been shown to be essential.

Suitable bioinformatics programs are well known to those skilled in the art and include the Basic Local Alignment Search Tool (BLAST) program (Altschul et al. (1990) J. Mol. Biol. 215: 403-410 and Altschul et al. (1997) Nucl. Acids Res. 25: 3389-3402). Suitable databases include, for example, EMBL, GENBANK, TIGR, EBI, SWISS-PROT and trEMBL.

Alternatively, or in addition, the (full or partial) sequence of the putative essential gene is used to interrogate a sequence database containing information as to the identity of essential genes which has been previously constructed using the conventional Tn-seq methods described in the prior art (e.g. as described in Gawronski et al. (2009) PNAS 106: 16422-16427; Goodman et al. (2009) Cell Host Microbe 6: 279-289; van Opijnen et al. (2009) Nat. Methods 6: 767-772; Langridge et al. (2009) Genome Research 19: 2308-2316; Gallagher et al. (2011) mBio 2(1):e00315-10) and/or the techniques described in WO 01/07651 (the contents of which are hereby incorporated by reference).

Alternatively, or in addition, essentiality can be imputed by eliminating the possibility that a putative essential gene acts as an antibiotic resistance gene. For example, the (full or partial) sequence of the putative essential gene is used to interrogate sequence databases containing sequence information of genes previously identified as antibiotic resistance genes using the Tn-seq methods described in e.g. Gawronski et al. (2009) PNAS 106: 16422-16427; Goodman et al. (2009) Cell Host Microbe 6: 279-289; Langridge et al. (2009) Genome Research 19: 2308-2316 or Gallagher et al. (2011) mBio 2(1):e00315-10. Antibiotic resistance genes may be identified in such methods as a class of niche-specific/conditionally essential genes.

Despite the presence of a promoter within the inserted sequence, many Tn_{A} insertions will disrupt gene/DNA function and allow identification of essential/important DNA regions, as in standard Tn-seq (including TraDIS). However, some transposons will be positioned appropriately with respect to specific important DNA regions, whereby transcription of those specific regions, driven by the inserted promoter, is enhanced significantly compared to endogenous transcription. By growing the mutant pool in increasing antibiotic concentrations and repeating the sequencing it is possible to observe changes in the number of reads, indicating not only which DNA region contributes to antibiotic survival, but also the relative contribution. The higher levels of specific antibiotic target transcription (driven by the transposon-inserted promoters) will favour bacterial survival in antibiotic and link insertion site to DNA region by proximity.

To identify the specific antibiotic target(s), the position of the inserted promoter can be assessed with respect to its contribution to increased transcription of relevant downstream DNA sequences. A mathematically/technically straightforward bioinformatics component of this technique permits recognition of the contribution of the inserted promoter sequence to transcription of the putative antibiotic target gene relative to data generated in the absence of the antibiotic. For example, transcription of the antibiotic target partial gene product may be enough to confer antibiotic resistance and bioinformatic analysis would allow an explanation. This information is observed in the number of specific mutants providing this advantage being greater than those observed in the absence of the antibiotic. In addition, the partial gene transcript may still encode enough information to allow translation of a truncated, but functional essential protein. Bioinformatics would allow the effects of transcriptional read through on genes downstream of the gene adjacent to the inserted transposon to be considered, where there is there no defined RNA transcription termination sequence.

For example, a transposon/promoter upstream of genes A, B and C may generate a polycistronic transcript of all three genes (A-C), upstream of B a polycistronic transcript of genes B and C and upstream of C just gene C. If the reads for the first two transposons were high and the third low in antibiotic then the antibiotic target would be gene B.

Microdroplet Encapsulation

The methods of the invention are suitable for high-throughput screening, since the methods involve compartmentalizing the screening assay in tiny volumes of growth medium in the form of discrete microdroplets. This permits each microdroplet to be treated as a separate culture vessel, permitting rapid screening of large numbers of individual liquid cultures using established microfluidic and/or cell-sorting methodologies.

Thus, the microdroplets of the invention function as individual, discrete culture vessels in which the target cells can be independently cultured in isolation from other mutants, analysed, manipulated, isolated and/or sorted.

Any suitable method may be employed for encapsulating the mutant target cells in microdroplets according to the invention. For example, the mutant target cells can be encapsulated in gel microdroplets according to the methods described in WO98/41869. Thus, gel droplets can be made using ionic or thermal gelling principles: typically, target cells are mixed with a fluid precursor of the gel matrix. This is then solidified by polymerization, causing as little trauma to the cells as possible: for example, shocks arising from changes in temperature, osmotic pressure and pH should be minimized. The use of hydrogels is generally preferred, since they exhibit high water retention and porosity, permitting free diffusion of nutrients and waste products. Many such matrices are known in the art, including polysaccharides, such as alginates, carageenans, agarose, chitosan, cellulose, pectins and polyacrylamides.

Target cells can be added to the gel matrix material to form a suspension and distributed evenly while the matrix is in liquid phase. Gel regions or gel droplets are formed by hardening of the matrix following the formation of small individual volumes of the gel matrix suspension droplets using established techniques. Suitable techniques include, for example, emulsification with oil, vortexing, sonication, homogenization, dropping using a syringe type apparatus, vibration of a nozzle attached to a reservoir of the suspension or atomization followed by electrostatic separation or cutting with rotating wires.

Emulsion Encapsulation

Preferred according to the invention is encapsulation by emulsification. As described below, both single and double-phase emulsions may be used, including water-in-oil (W/O) type and water-in-oil-in water (W/O/W) type emulsions. In W/O emulsions, the dispersed phase may comprise microdroplets of aqueous growth medium suspended in a continuous oil phase. In W/O/W emulsions, the dispersed phase may comprise microdroplets of aqueous growth medium enveloped in an oil phase, which microdroplets are in turn suspended in a continuous aqueous phase. Such W/O/W emulsions may exhibit improved rheological properties over simple W/O emulsions: they may for example exhibit lower viscosity, permitting higher flow rates/lower running pressures when sorted using FADS and/or processed using microfluidic devices (see below).

Thus, in the simplest embodiments of the invention, encapsulation involves the formation of emulsions comprising microdroplets of liquid growth medium containing mutant target cells dispersed in a carrier liquid as the continuous phase. Such single emulsions are of the W/O type, though it will be appreciated that any suitable liquid may be used as the continuous phase provided that it is immiscible with the growth medium selected for culture of the target cells. Typically, however, the carrier liquid is an oil.

Depending inter alia on the nature of the continuous phase (e.g. the type of oil) and the droplet size, single emulsions of the type described above may have a viscosity that makes sorting of the microdroplets by certain microfluidic and/or cell sorting techniques difficult. For example, the viscosity may limit the flow rates achievable (e.g. to the range of 10-100 microdroplets per second using FADS—see below) and/or may require undesirably high operating pressures.

Double Emulsions

Particularly preferred according to the invention are double emulsions of the W/O/W type. Such emulsions comprise microdroplets containing an aqueous core of liquid aqueous growth medium containing mutant target cells enveloped in a shell of immiscible liquid (typically an oil), these microdroplets being dispersed in an aqueous carrier liquid as the continuous phase.

Cultures encapsulated in this way can exhibit very low viscosities, and can therefore be sorted using e.g. FADS (see below) at much higher flow rates, permitting sorting of over 10,000 microdroplets per second (see e.g. Bernath et al. (2004) Analytical Biochemistry 325: 151-157).

Encapsulation in double emulsions may be achieved by any of a wide variety of techniques, using a wide range of suitable aqueous growth media, carrier oils and surfactants. Suitable techniques for making double emulsions (and for sorting them using FACS) is described, for example, in Bernath et al. (2004) Analytical Biochemistry 325: 151-157.

Surfactants for Use in Emulsion Encapsulation

As explained herein, the microdroplets (and the corresponding microcultures) of the invention may comprise a single water-in-oil (W/O) or double water-in-oil-in-water (W/O/W) type emulsions, and in such embodiments one or more surfactants may be necessary to stabilize the emulsion.

The surfactant(s) and/or co-surfactant(s) are preferably incorporated into the W/O interface(s), so that in embodiments where single W/O type emulsions are used the surfactant(s) and or co-surfactant(s) may be present in at the interface of the aqueous growth medium microdroplets and the continuous (e.g., oil) phase. Similarly, where double W/O/W type emulsions are used for encapsulation according to the invention, the surfactant(s) and or co-surfactant(s) may be present at either or both of the interfaces of the aqueous core and the immiscible (e.g. oil) shell and the interface between the oil shell and the continuous aqueous phase.

A wide range of suitable surfactants are available, and those skilled in the art will be able to select an appropriate surfactant (and co-surfactant, if necessary) according to the selected screening parameters. For example, suitable surfactants are described in Bernath et al. (2004) Analytical Biochemistry 325: 151-157; Holtze and Weitz (2008) Lab Chip 8(10): 1632-1639; and Holtze et al. (2008) Lab Chip. 8(10):1632-1639.

The surfactant(s) are preferably biocompatible. For example, the surfactant(s) may be selected to be non-toxic to the mutant target cells. The selected surfactant(s) may also have good solubility for gases, which may be necessary for the growth and/or viability of the encapsulated cells.

Biocompatibility may be determined by any suitable assay, including assays based on tests for compatibility with a reference sensitive biochemical assay (such as in vitro translation) which serves as a surrogate for biocompatibility at the cellular level. For example, in vitro translation (IVT) of plasmid DNA encoding the enzyme β-galactosidase with a fluorogenic substrate (fluorescein di-β-D-galactopyranoside (FDG)) may be used as an indicator of biocompatibility since a fluorescent product is formed when the encapsulated DNA, the molecules involved in transcription and translation, and the translated protein do not adsorb to the drop interface and the higher-order structure of the protein remains intact.

The surfactant(s) may also prevent the adsorption of biomolecules at the microdroplet interface. This may contribute to biocompatibility, e.g. by preventing sequestration of biomolecules necessary for cellular growth, gene expression and/or signalling.

The surfactant may also function to isolate the individual microdroplets (and the corresponding microcultures), so that they serve as individual microvessels for culture of mutant target cells. In such embodiments, the surfactant may be both hydrophobic and lipophobic, and so exhibit low solubility for the biological reagents of the aqueous phase while inhibiting molecular diffusion between microdroplets.

In some embodiments, the surfactant stabilizes (i.e. prevents coalescence) of a single emulsion comprised of droplets of aqueous media (containing encapsulated cells) dispersed in an oil phase for the duration of the incubation step. Similarly, the surfactant may stabilize droplets of aqueous media (containing encapsulated cells) in a double water-in-oil-in-water (W/O/W) type emulsion for the duration of the incubation step.

Thus, the surfactant may stabilize the microdroplet library under the conditions employed for culture of the single mutant target cells, and so may stabilize the microdroplet at the selected incubation temperature (e.g. about 37° C.) for the selected incubation time (e.g. for at least an hour, and in preferred embodiments for up to 14 days).

Stabilization performance can be monitored by e.g. phase-contrast microscopy, light scattering, focused beam reflectance measurement, centrifugation and/or rheology.

Examples of suitable surfactants include: Abil WE 09 (Evonik—a 1:1:1 mixture of cetyl PEG/PPG 10/1 dimethicone, polyglyceryl-4 isostearate and hexyl laurate); Span® 80; Tween® 20; Tween® 80 and combinations thereof.

Oils for Use in Emulsion Encapsulation

While it will be appreciated than any liquid immiscible with the aqueous growth medium may be used in the formation of microdroplet emulsions for use according to the invention, the immiscible fluid is typically an oil.

Preferably, an oil is selected having low solubility for biological components of the aqueous phase. Other preferred functional properties include good solubility for gases, the ability to inhibit molecular diffusion between microdroplets and/or combined hydrophobicity and lipophobicity. The oil may be a hydrocarbon oil, but preferred are light mineral oils, fluorocarbon or ester oils. Mixtures of two or more of the above-described oils are also preferred.

Examples of suitable oils include: diethylhexyl carbonate (Tegosoft® DEC (Evonik)); light mineral oil (Fisher); and combinations thereof.

Processes for Microdroplet Emulsification

A wide range of different emulsification methods are known to those skilled in the art, any of which may be used to create the microdroplets of the invention.

Many emulsification techniques involve mixing two liquids in bulk processes, often using turbulence to enhance drop breakup. Such methods include vortexing, sonication, homogenization or combinations thereof.

In these “top-down” approaches to emulsification, little control over the formation of individual droplets is available, and a broad distribution of microdroplet sizes is typically produced. Alternative “bottom up” approaches operate at the level of individual drops, and may involve the use of microfluidic devices. For example, emulsions can be formed in a microfluidic device by colliding an oil stream and a water stream at a T-shaped junction: the resulting microdroplets vary in size depending on the flow rate in each stream.

A preferred process for producing microdroplets for use according to the invention comprises flow focusing (as described in e.g. Anna et al. (2003) Appl. Phys. Lett. 82(3): 364-366). Here, a continuous phase fluid (focusing or sheath fluid) flanking or surrounding the dispersed phase (focused or core fluid), produces droplet break-off in the vicinity of an orifice through which both fluids are extruded. A flow focusing device consists of a pressure chamber pressurized with a continuous focusing fluid supply. Inside, one or more focused fluids are injected through a capillary feed tube whose extremity opens up in front of a small orifice linking the pressure chamber with the external ambient environment. The focusing fluid stream moulds the fluid meniscus into a cusp giving rise to a steady micro or nano-jet exiting the chamber through the orifice; the jet size is much smaller than the exit orifice. Capillary instability breaks up the steady jet into homogeneous droplets or bubbles.

The feed tube may be composed of two or more concentric needles and different immiscible liquids or gases be injected leading to compound drops. Flow focusing ensures an extremely fast as well as controlled production of up to millions of droplets per second as the jet breaks up.

Other possible microfluidic droplet-forming techniques include pico-injection, whereby oil in water droplets are first formed and then pass down a T junction, along the top of the T and then inner aqueous phase is injected into the oil droplet creating a double emulsion.

In all cases, the performance of the selected microdroplet forming process may be monitored by phase-contrast microscopy, light scattering, focused beam reflectance measurement, centrifugation and/or rheology.

Fluorescence-Activated Droplet Sorting

As explained herein, the methods of invention are suitable for high-throughput screening, since they involve compartmentalizing the screening assay in tiny volumes of growth medium in the form of discrete microdroplets. This permits each microdroplet to be treated as a separate culture vessel, permitting rapid screening of large numbers of individual liquid cultures using established microfluidic and/or cell-sorting methodologies.

Thus, following the encapsulation steps, the resultant microdroplets may be sorted by adapting well-established fluorescence-activated cell sorting (FACS) devices and protocols. This technique has been termed Fluorescence-Activated Droplet Sorting (FADS), and is described, for example, in Baret et al. (2009) Lab Chip 9: 1850-1858.

FADS may be used to manipulate the microdroplets at any stage after their formation in the methods of the invention. FADS may also be used during the encapsulation steps, for example to eliminate empty microdroplets which do not contain mutant target cells.

The mutant target cells may be fluorescently labelled to enable FADS. A variety of fluorescent proteins can be used as labels for this purpose, including for example the wild type green fluorescent protein (GFP) of Aequorea victoria (Chalfie et al. 1994, Science 263:802-805), and modified GFPs (Heim et al. 1995, Nature 373:663-4; PCT publication WO 96/23810). Alternatively, DNA2.0's IP-Free© synthetic non-Aequorea fluorescent proteins can be used as a source of different fluorescent protein coding sequences that can be amplified by PCR or easily excised using the flanking Bsal restriction sites and cloned into any other expression vector of choice.

Transcription and translation of this type of reporter gene leads to accumulation of the fluorescent protein in the cells, so rendering them amenable to FADS.

Incubation

The incubation conditions and the nature of the aqueous growth medium are selected according to the nature of the selected target cells, and those skilled in the art will be able to readily determine appropriate media, growth temperatures and duration of incubation.

Preferably, the microcultures are incubated at between 25° C. and 42° C. (for example, about 37° C.) The microcultures may be maintained at said temperature for about 2, 4, 6, 12, 24 or 48 hours. In preferred embodiments, the microcultures are grown for a sufficient time to permit substantially all microcultures to reach the post-exponential growth phase, and preferably for long enough to reach the stationary growth phase. For example, the incubation step may comprise maintaining the microdroplet libraries at said temperature for up to 7 days, for example for 1, 2, 3, 4, 5, 6 or 7 days.

However, depending on the target bacterium selected and the nature of the growth medium (and in particular, whether the growth medium is a rich medium or a defined, minimal medium), the incubation step may comprise maintaining the microdroplet libraries at said temperature for over 7 days, for example up to 10 days or 2 weeks, for example for 1 week or 2 weeks.

The incubation step may be conducted under conditions such that the each mutant cell contained in each microdroplet undergoes a number of doublings sufficient to yield 100-10,000 cells, preferably 500-5000, and most preferably about 1000 cells.

### EXEMPLIFICATION

The invention will now be described with reference to specific Examples. These are merely exemplary and for illustrative purposes only: they are not intended to be limiting in any way to the scope of the monopoly claimed or to the invention described. These examples constitute the best mode currently contemplated for practicing the invention.

### Example 1: Encapsulation Using Single Emulsion

Oil Mix for Continuous Phase
73% Tegosoft DEC (Evonik), 20% light mineral oil (Fisher), 7% Abil WE09 (Evonik) (surfactant)
70% Tegosoft DEC (Evonik), 20.3% light mineral oil (Fisher) 4.5% Span-80 (surfactant), 4.8% Tween20 (surfactant)
90% light mineral oil, 10% Span-80 (surfactant)

All oil mixes need to be made at least 30 minutes prior to use but can be kept indefinitely.

Aqueous Growth Media for Dispersed Phase

This may be selected from the following:
SOC broth (20 g/L tryptone, 5 g/L yeast extract 10 mM NaCl, 2.5 mM KCl, 10 mM MgCl₂, 10 mM MgSO₄ and 20 mM glucose)
SOC+5% Glycerol
LB broth (10 g/L Peptone, 5 g/L Yeast extract, 10 g/L NaCl)
2×YT (16 g/L tryptone, 10 g/L Yeast Extract, 5 g/NaCl)
2×YT+0.5% Glucose and 5% Gylcerol
2×YT+5% glycerol
Peptone glycerol media 5 g/L peptone, 5% glycerol

The inclusion of glycerol as a carbon source can facilitate microdroplet formation by vortexing.
1. 0.5 ml to 10 ml of growth media containing the target cells at appropriate cell numbers to give <1 target cell per droplet produced. This is aliquoted into a suitable vessel (Eppendorf tube 1.5 or 2 ml 15 ml or 50 ml falcon or 24 well plate are acceptable).
2. This growth media is then overlaid with 1-2× volume of the oil mix.
3. This is the vortexed for between 3-6 minutes (usually 5 minutes) at 12,000-18,000 rpm. The speed varies by oil and media used as well as the desired droplet size as a general rule the higher the speed and the longer the vortexing the smaller the average droplet size (although a range of sizes are always generated by this method).
4. The integrity of the droplets and presence of cells within is screened visually under a microscope using phase contrast. Alternative visualisation methods can be used e.g. fluorescence.
5. Droplets are then incubated at appropriate temperature (usually 37° C., but they are stable between 4° C. and 95° C.) for time periods of 1 h to ≧14 days.
6. Droplets can then be sorted by FADS if required and then desired cells recovered from the droplets.
7. To break open droplets additional surfactant is added (e.g. 1% SDS for Tegosoft oil, but any appropriate surfactant such as Tweens, Sarcosyl etc. can be used) this disrupts droplet integrity releasing cells.
8. Some oil mixes can be lysed by freezing the solution disrupting the droplets.
9. Cells can then be recovered by centrifugation, or the sample can be applied directly to columns for DNA extractions.

### Example 2: Encapsulation Using Double Emulsion

Aqueous Growth Media

As in Example 1 (above).

Mixes Used to Produce Outer Aqueous Phase
Phosphate buffered Saline (PBS; 10 mM phosphate buffer, 137 mM NaCl) with 2% Tween-20
PBS with 2% Tween-80 (surfactant)

Oil Mix for Droplet Shells

As for the oil mixes set out in Example 1 (above).
1. 0.5 ml to 10 ml of growth media containing the target cells at appropriate cell numbers to give <1 target cell per droplet produced. This is aliquoted into a suitable vessel (Eppendorf tube 1.5 or 2 ml 15 ml or 50 ml falcon or 24 well plate are acceptable).
2. This growth media is then overlaid with 1-2× volume of the oil mix.
3. This is the vortexed for between 3-6 minutes (usually 5 minutes) at 12,000-18,000 rpm. The speed varies by oil and media used as well as the desired droplet size as a general rule the higher the speed and the longer the vortexing the smaller the average droplet size although a range of sizes are always generated by this method.
4. The integrity of the droplets and presence of cells within is screened visually under a microscope using phase contrast. Alternative visualisation methods can be used e.g. fluorescence.
5. A second vortexing step is then introduced to generate the double emulsion. A volume equal to the oil volume of second aqueous phase with surfactant is added to the single emulsion. This is then vortexed as but at reduce rpm (6,000 to 10,000 rpm) for just 2-3 minutes. The formation of double emulsion droplets can then be visualised on a microscope as before.
6. Droplets are then incubated at appropriate temperature (usually 37° C., but they are stable between 4° C. and 95° C.) for time periods of 1 h to ≧14 days.
7. Droplets can then be sorted by FADS if required and then desired cells recovered from the droplets.
8. To break open droplets additional surfactant is added (e.g. 1% SDS for Tegosoft oil, but any appropriate surfactant such as Tweens, Sarcosyl etc can be used) this disrupts droplet integrity releasing cells.
9. Some oil mixes can be lysed by freezing the solution disrupting the droplets.
10. Cells can then be recovered by centrifugation, or the sample can be applied directly to columns for DNA extractions.

### Example 3: Production of Microdroplets Using a Microfluidic Chip

Droplet generation in a microfluidic allows creation of single and double emulsions. In its simplest form double emulsions are formed by sequential formation of and oil in water droplet and then formation of a second aqueous layer by the same methodology.

Alternative methods involve simultaneous encapsulation of aqueous phase in oil and then the oil-aqueous droplet in a secondary continuous phase of aqueous media. Droplet formation on a chip leads to generation of highly uniform sized droplets. Size is directly related to the size of the channels on the fluidic and the flow rates used to generate droplets. Microfludic droplet formation allows the use of novel oil and surfactant mixes not available when producing droplets in bulk “top-down” approaches.

The target cells in suitable growth media are mixed at appropriate ratios to allow for ≦1 target per droplet. This aqueous mixture of cells is then pumped through a microfluidic device with geometry that allows the formation of water in oil single emulsion droplets, or double emulsion droplets, as detailed below.

These droplets are collected in a vessel suitable for incubation and the incubated at appropriate temperature (usually 37° C., but they are stable between 4° C. and 95° C.) for time periods of 1 h to ≧14 days.

Droplets can then be sorted by FADS if required and then desired cells recovered from the droplets.

To break open droplets, additional surfactant is added (e.g. 1% SDS for Tegosoft oil, but any appropriate surfactant such as Tweens, Sarcosyl etc. can be used) to disrupt droplet integrity, so releasing the cells. Some oil mixes can be lysed by freezing the solution disrupting the droplets.

Cells can then be recovered by centrifugation, or the sample can be applied directly to columns for DNA extractions.

### Example 4: Production of Double Emulsion Microdroplets Using Flow Focusing

Here, oil flows from the sides into a hydrophobic channel causing pinch off of the aqueous phase generating the water in oil droplets. This method allows good size control and 1000's of droplets per second to be generated depending on flow rates of the second liquid phases.

It is possible to use two such chips connected in series to generate double emulsions: the first droplets are aqueous phase in oil which are pushed through a second chip as if aqueous phase, the side flowing oil being replaced with the secondary aqueous phase.

It is also possible to integrate both steps into a single chip and junction interface; whereby water in oil droplets are formed and immediately surrounded in the outer aqueous phase to generate the W/O/W double emulsion.

The hydrophilic and hydrophobic coatings are inverted on the second chip, thus the second chip has the same geometry, but the opposite surface coatings to facilitate flow of aqueous phase as the carrier/sheath fluid. This permits a two stage encapsulation: the first to generate single phase emulsions which are used for incubation, and a second stage in which the microcultures are converted into double emulsions for subsequent FADS.

### Example 5: Microcultures Eliminate Signal Loss Arising from Out-Competition by Fast-Growing Mutants

Mutant bacteria produced as described in WO2012/150432 contain TnA insertions in genes which mediate antibiotic sensitivity and/or resistance. The individual mutants display a wide range of fitness, to the extent that conventional co-culture of the mutants in bulk liquid culture results in surprisingly high losses of antibiotic resistant mutants of interest which are “swamped” by faster growing mutants.

Culturing each mutant in its own discrete micro-environment using the microdroplets of the invention effectively eliminates signal loss arising from out-competition by faster growing mutants, greatly increasing the resolution of the technology.

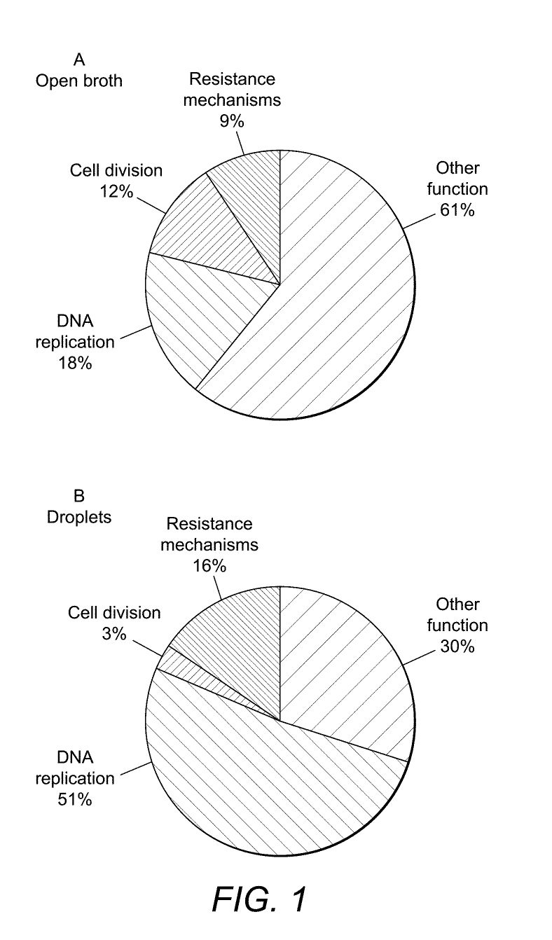

FIG. 1 shows the results of exposure to ciprofloxacin (EC95) in E. coli cells mutagenized with an activating transposon (TnA) as described in WO2012/150432. The bactericidal action of ciprofloxacin results from inhibition of the enzymes topoisomerase II (DNA gyrase) and topoisomerase IV (both Type II topoisomerases), which are required for bacterial DNA replication, transcription, repair and recombination.

Sequencing of the TnA insertion sites revealed genes which, when upregulated, increase fitness in the presence of ciprofloxacin. These genes were then grouped into four classes: (a) genes involved in resistance mechanisms; (b) genes involved in cell division; (c) genes involved in DNA replication; and (d) genes involved in other cellular processes.

The data shows the proportions of the four different mutant classes (when grown in standard open broth culture (FIG. 1, Part A) and in microdroplets prepared as described in Example 1 (FIG. 1, Part B). As is apparent from these data, only 18% of genes identified as activated during growth in open culture were directly associated with the mechanism of action (DNA replication), whereas when the TnA mutants were grown in microdroplet culture, 51% of genes were directly associated with the mechanism of action.

Thus, culturing the mutants in discrete microcultures effectively eliminates signal loss arising from out-competition by faster growing mutants, greatly increasing the resolution of the technology.

### EQUIVALENTS

The foregoing description details presently preferred embodiments of the present invention. Numerous modifications and variations in practice thereof are expected to occur to those skilled in the art upon consideration of these descriptions. Those modifications and variations are intended to be encompassed within the claims appended hereto.

1. A method for identifying a gene which mediates antibiotic sensitivity or resistance in a target bacterium, the method comprising the steps of:
(a) generating a pool of mutant target bacteria by transposon mutagenesis with an activating transposon (TnA), wherein the TnA comprises an outward-facing promoter (TnAP) capable of increasing transcription of a gene at or near its insertion site in the DNA of said target cells;
(b) generating a control microdroplet library by encapsulating individual members of the pool of step (a) in microdroplets, the microdroplets comprising a volume of aqueous growth media suspended in an immiscible carrier liquid, each microdroplet comprising a single mutant target cell;
(c) generating a test microdroplet library by encapsulating individual members of the pool of step (a) in microdroplets, the microdroplets comprising a volume of aqueous growth media containing the antibiotic and suspended in an immiscible carrier liquid, each microdroplet comprising a single mutant target cell;
(d) incubating the control and test microdroplet libraries to produce control and test microcultures; and
(e) comparing the distribution of TnA insertions between control and test microcultures to identify a gene which mediates antibiotic sensitivity or resistance in said target bacterium.

2. The method of claim 1 wherein the test microdroplet library comprises microdroplets containing the antibiotic at a concentration of: (a) about 0.5; (b) about 1; or (c) about 2×MIC.

3. The method of claim 1 wherein a plurality of test microdroplet libraries are generated, each containing a different concentration of antibiotic.

4. The method of claim 1 wherein the pool of mutant bacteria comprises: (a) at least 0.5×105 mutants, for example at least 1×105 mutants; (b) at least 5×105 mutants; (c) at least 1×106 mutants; (d) 0.5×106 to 2×106 mutants; (e) about 1×106 mutants; or (f) up to 10×106 mutants.

5. The method of claim 1 wherein the transposon mutagenesis step (a) yields an insertion rate of: (a) at least one transposon per 50 base pairs of bacterial DNA; (b) at least one transposon per 30 base pairs of bacterial DNA; (c) at least one transposon per 25 base pairs of bacterial DNA; (d) at least one transposon per 15 base pairs of bacterial DNA; (e) at least one transposon per 10 base pairs of bacterial DNA; or (f) at least one transposon per 5 base pairs of bacteria DNA.

6. The method of claim 1 wherein in step (a) said DNA of the bacterium is (i) chromosomal (genomic) DNA; (ii) plasmid DNA; or (ii) a mixture of chromosomal (genomic) and plasmid DNA.

7. (canceled)

8. The method of claim 1 wherein the transposon mutagenesis of step (a) occurs in vivo or in vitro.

9. (canceled)

10. The method of claim 1 wherein the distribution of TnA insertions between control and test cultures is compared by identifying: (a) the insertion position in the genome; and/or (b) the abundance of each insertion in the genome.

11. The method of claim 1 wherein the distribution of TnA insertions between control and test cultures is compared by a method comprising sequencing DNA adjacent or near the insertion site of the TnA.

12. The method of claim 11 wherein the sequencing of DNA adjacent or near the insertion site of the TnA comprises selective amplification of transposon-bacterial DNA junctions.

13-15. (canceled)

16. The method of claim 1 further comprising the step of sequencing mRNA transcripts produced by TnAP in mutant target cells to produce an mRNA transcript profile.

17-20. (canceled)

21. The method of claim 1 wherein the microdroplets comprise an inner core of aqueous growth media enveloped in an outer oil shell, the carrier liquid being a continuous aqueous phase.

22-37. (canceled)

38. The method of claim 1 wherein the encapsulation steps (b) and (c) comprise mixing: (a) the pool of mutant target cells; (b) an aqueous growth medium; (c) a water-immiscible liquid and (d) a surfactant, under conditions whereby a W/O type single emulsion comprising microdroplets of the aqueous growth medium dispersed in the water-immiscible liquid is formed.

39-40. (canceled)

41. The method of claim 38 wherein the mixing comprises: (a) vortexing and/or (b) sonication; (c) homogenization; (d) pico-injection and/or (e) flow focusing.

42-43. (canceled)

44. The method of claim 1 wherein incubation step (d) comprises maintaining the microdroplet libraries at a temperature of 15° C.-42° C. for at least 1 hour.

45-48. (canceled)

49. The method of claim 1 wherein the target bacterium is a pathogenic bacterium.

50-55. (canceled)

56. A method of identifying an antibiotic comprising identifying a gene which mediates antibiotic sensitivity or resistance in a target bacterium according to a method as defined in claim 1.

57. A process for producing an antibiotic comprising the method as defined in claim 1.

58-59. (canceled)
